(19) United States
(12) Patent Application Publication
(10) Pub. No.: US 2022/0057274 A1
(43) Pub. Date: Feb. 24, 2022

(54) PATCH TYPE THERMOMETER AND APPLICATION PRODUCT FOR SAME

(71) Applicant: NBST Co., Ltd., Pyeongtaek-si (KR)

(72) Inventors: In Suk CHO, Pyeongtaek-si (KR); Gwang Ho KIM, Pyeongtaek-si (KR); In sook LEE, Pyeongtaek-si (KR); Seok Hyoung KIM, Pyeongtaek-si (KR)

(21) Appl. No.: 16/952,270

(22) Filed: Nov. 19, 2020

(30) Foreign Application Priority Data
Aug. 24, 2020 (KR) 10-2020-0106446

(51) Int. Cl. G01K 11/16; A61B 5/00; C09D 11/50; A61B 5/01

(52) U.S. Cl. CPC G01K 11/16; A61B 5/6833; C09D 11/50; A61B 2503/08; A61B 5/01; A61B 2503/04; A61B 2503/06; A61B 5/68335

(57) ABSTRACT

The present disclosure relates to a patch-type thermometer using a thermochromic ink, and particularly to, a patch-type thermometer capable of accurately measuring a body temperature even in special environments.

### CROSS-REFERENCE TO RELATED APPLICATION

This application claims priority to Korean Patent Application No. 10-2020-0106446 filed on Aug. 24, 2020 and all the benefits accruing therefrom under 35 U.S.C. § 119, the contents of which are incorporated by reference in their entirety.

### BACKGROUND

The present disclosure relates to a patch-type thermometer and applications thereof, and more particularly, to a patch-type thermometer capable of accurately measuring a body temperature in various environments.

Infectious diseases such as corona virus (COVID-19) or swine flu, which are spreading these days, are often accompanied by a high fever. In the case of infectious diseases, if a person is not diagnosed and quarantined early, there is a concern of large-scale spread (pandemic). Thus, at a time when the infectious diseases are prevalent, there is a need for a thermometer which can easily and quickly determine a high fever when a person's body temperature exceeds a normal body temperature.

However, it is difficult for parents to monitor the physical conditions of infants or toddlers because the infants or infants frequent move or resist, making it difficult to continuously measure the body temperatures thereof. It is also difficult for the elderly to actively cope with changes in their body temperature. Therefore, there is a need for a thermometer which can easily, quickly and accurately measure the body temperatures of infants, toddlers, the elderly, and the like.

A typical thermometer is contacted with a subject's body for a certain period of time, and then separated from the same to check the body temperature of the subject, so it is inconvenient to check a body temperature in real time or periodically. There is also a typical thermometer which is attachable to a person's body, making it easy to measure a body temperature, but since the thermometer is thick, it is inconvenient to keep the thermometer attached to the body, and there is also a problem in that, in a special environment (e.g., when infants and toddlers play in the water, or in the summer when people sweat a lot, and the like), the measurement of body temperature is not relatively accurate.

### SUMMARY

The present disclosure provides a patch-type thermometer capable of relatively accurately measuring a body temperature without being affected by the external environment even in a variety of environments.

The present disclosure also provides a patch-type thermometer which makes it possible to determine whether or not a person has a high fever using colors displayed, so that even infants and toddlers, who do not have a concept of temperature, or the elderly become aware of danger to be able to quickly respond to infectious diseases.

The present disclosure also provides a body temperature measuring band product using a patch-type thermometer.

In accordance with an exemplary embodiment of the present invention, a patch-type thermometer includes a first film layer, and a thermochromic ink composition mixed layer stacked on the first film layer and composed of a thermochromic ink composition including first thermochromic ink powder exhibiting a first color, second thermochromic ink powder exhibiting a second color, third thermochromic ink powder exhibiting a third color, and a binder.

Particularly, the patch-type thermometer may have the configuration of a first form of patch-type thermometer further including an adhesive layer stacked on a lower side of the first film layer and a lower release layer stacked on a lower side of the adhesive layer in addition to the above basic configuration.

Meanwhile, the patch-type thermometer may have the configuration of a second form of patch-type thermometer further including a lower release layer, a second film layer, an adhesive layer, and an upper release layer in addition to the above basic configuration. Specifically, the patch-type thermometer may further include a lower release layer, a first film layer stacked on the lower release layer, a thermochromic ink composition mixed layer stacked on the first film layer and composed of a thermochromic ink composition including first thermochromic ink powder exhibiting a first color, second thermochromic ink powder exhibiting a second color, third thermochromic ink powder exhibiting a third color, and a binder, an adhesive layer stacked on an upper side of the thermochromic ink composition mixed layer, a second film layer stacked on an upper side of the adhesive layer, and an upper release layer stacked on an upper side of the second film layer.

At this time, the upper release layer and/or the lower release layer may preferably contain one or more components selected from polyester (PET), polyethylene (PE), polypropylene (PP), and paper, the first film layer may preferably contain one or more components selected from an Oriented Polypropylene (OPP) film, PET, PP, and paper, and the second film layer may preferably contain one or more components selected from urethane, PE, and PP.

Here, the paper may be oil diffusion paper or art paper.

The thermochromic ink composition mixed layer may be prepared by adding Thermo Dye Ink powders and a binder to a composition containing a melamine copolymer, styrene maleic anhydride, methyl ester, and ethyl ester. Dyes and the mixing ratio thereof of the Thermo Dye Ink powders may be appropriately changed according to an exemplary embodiment of the present invention, that is, depending on the color to be displayed according to a temperature. For example, according to an embodiment of the present invention, three types of Thermo Dye Ink powders, which respectively exhibit magenta, cyan, and yellow colors, may be prepared. The thermochromic ink composition mixed layer of the present invention may be a mixture of dyes, and thus, may correspond to 1-degree printing.

At this time, the thermochromic ink composition mixed layer may exhibit the third color in the case of a low body temperature, may exhibit the second color in the case of a normal body temperature, and may exhibit the first color in the case of a body temperature higher than the normal body temperature.

Meanwhile, preferably, based on the total weight of the thermochromic ink powder, the first thermochromic ink powder exhibiting the first color may be contained in a range of approximately 20 wt % to approximately 40 wt %, the second thermochromic ink powder exhibiting the second color may be contained in a range of approximately 40 wt % to approximately 60 wt %, the third thermochromic ink powder exhibiting the third color may be contained in a range of approximately 20 wt % to approximately 40 wt %, or more preferably, the first thermochromic ink powder exhibiting the first color may be contained in a range of approximately 25 wt % to approximately 35 wt %, the second thermochromic ink powder exhibiting the second color may be contained in a range of approximately 45 wt % to approximately 55 wt %, the third thermochromic ink powder exhibiting the third color may be contained in a range of 25 wt % to approximately 35 wt %.

The second film layer may have a waterproof function.

The thermochromic ink composition mixed layer included in the patch-type thermometer of the present invention may exhibit different colors depending on the temperature. According to an embodiment of the present invention, the patch-type thermometer may exhibit the third color in the case of a low body temperature or before being attached to a human body (lower than or equal to approximately 35° C.), may exhibit a mixed color of the first and second colors below approximately 37.5° C., which is a normal body temperature of a person, with the third color disappearing, and may exhibit only the first color in the case of a body temperature higher than or equal to approximately 37.5° C., with the second color disappearing.

### BRIEF DESCRIPTION OF THE DRAWINGS

Exemplary embodiments can be understood in more detail from the following description taken in conjunction with the accompanying drawings, in which:

FIG. 1 shows the stacked structure of a patch-type thermometer in accordance with an exemplary embodiment of the present invention;

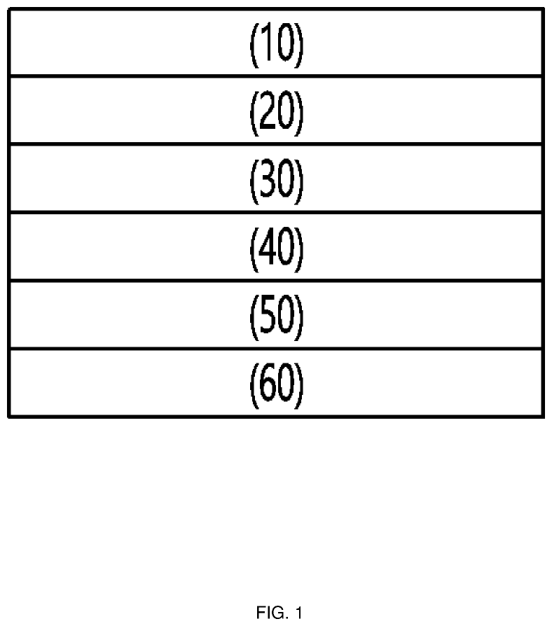

FIG. 2 shows the color of a thermochromic ink composition of a patch-type thermometer in accordance with another exemplary embodiment of the present invention;

FIG. 3 shows the temperature display unit of a patch-type thermometer in accordance with yet another exemplary embodiment of the present invention;

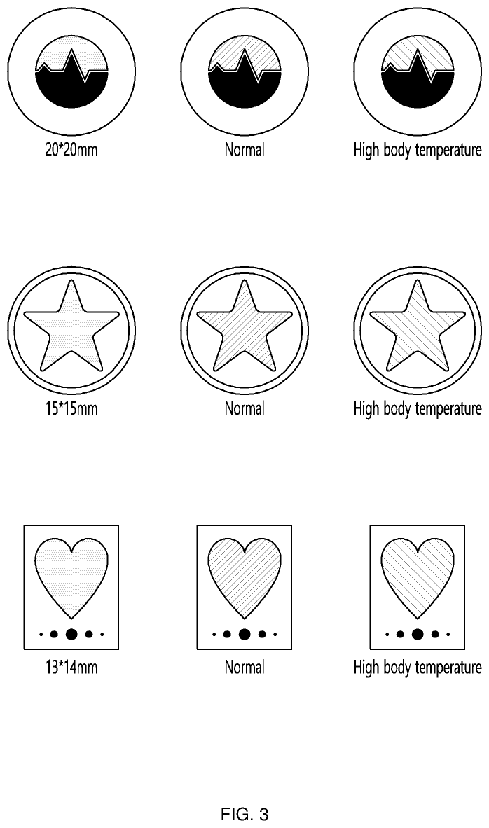

FIG. 4 shows an embodiment of a patch-type thermometer including a release layer in accordance with still another exemplary embodiment of the present invention; and

FIG. 5 shows a method for using a patch-type thermometer in accordance with yet still another exemplary embodiment of the present invention.

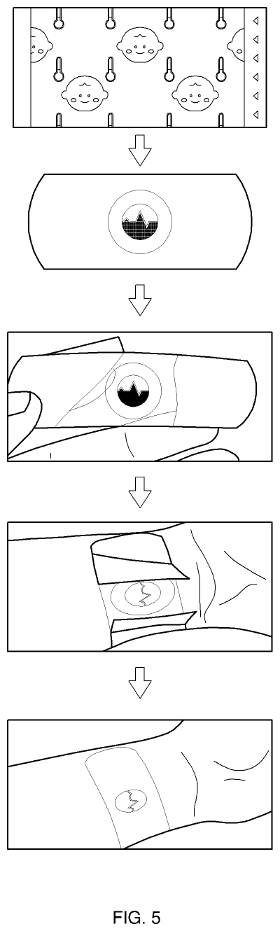

### DETAILED DESCRIPTION OF EMBODIMENTS

Examples of a body temperature measurement patch and its application products according to the present invention may be applied in various ways, and are not to be construed as being limited to the following examples, but to include all modifications, equivalents, and alternatives included in the spirit and scope of the present invention.

The present invention relates to a patch-type thermometer capable of easily and quickly check a body temperature by using a thermochromic ink. The patch-type thermometer of the present invention includes a first film layer, and a thermochromic ink composition mixed layer composed of a thermochromic ink composition including first thermochromic ink powder exhibiting a first color, second thermochromic ink powder exhibiting a second color, third thermochromic ink powder exhibiting a third color, and a binder, and stacked on the first film layer.

Particularly, the patch-type thermometer of the present invention may have the configuration of a first form of patch-type thermometer further including an adhesive layer stacked on a lower side of the first film layer and a lower release layer stacked on a lower side of the adhesive layer in addition to the above basic configuration.

Meanwhile, the patch-type thermometer of the present invention may have the configuration of a second form of patch-type thermometer further including a lower release layer, a second film layer, an adhesive layer, and an upper release layer in addition to the above basic configuration. Specifically, the patch-type thermometer further includes a lower release layer, a first film layer stacked on the lower release layer, a thermochromic ink composition mixed layer stacked on the first film layer and composed of a thermochromic ink composition including first thermochromic ink powder exhibiting a first color, second thermochromic ink powder exhibiting a second color, third thermochromic ink powder exhibiting a third color, and a binder, an adhesive layer stacked on an upper side of the thermochromic ink composition mixed layer, a second film layer stacked on an upper side of the adhesive layer, and an upper release layer stacked on an upper side of the second film layer.

According to an embodiment of the present invention, the first film layer has flexibility, and thus, the present patch-type thermometer improves the feeling of use when attached. The first film layer is preferably an OPP film layer composed of a polypropylene component, but one or more components selected from PET, PE, and paper may be also used. Here, the thickness of the first film layer is preferably in the range of approximately 30 μm to approximately 100 μm.

According to an embodiment of the present invention, the thermochromic ink composition mixed layer contains thermochromic ink powder which changes its color in a certain temperature range in order to determine a normal body temperature and a high fever. The thermochromic ink composition mixed layer includes thermochromic ink powders which may respectively exhibit the first color, the second color, and the third color at a certain temperature and a binder in a certain ratio, and is formed or stacked on an upper side of the first film layer to form one layer. Here, the thickness of the thermochromic ink composition mixed layer is preferably in the range of approximately 10 μm to approximately 80 μm.

According to an embodiment of the present invention, based on the total weight of the thermochromic ink powder, the first thermochromic ink powder exhibiting the first color is contained in a range of approximately 20 wt % to approximately 40 wt %, the second thermochromic ink powder exhibiting the second color is contained in a range of approximately 40 wt % to approximately 60 wt %, the third thermochromic ink powder exhibiting the third color may be included in a range of approximately 20 wt % to approximately 40 wt %, and preferably, the first thermochromic ink powder exhibiting the first color is contained in a range of approximately 25 wt % to approximately 35 wt %, the second thermochromic ink powder exhibiting the second color may be included in a range of approximately 45 wt % to approximately 55 wt %, the third thermochromic ink powder exhibiting the third color is contained in a range of approximately 25 wt % to approximately 35 wt %. More preferably, the first thermochromic ink powder is contained at a ratio of approximately 25 wt %, the second thermochromic ink powder is contained at a ratio of approximately 50 wt %, the third thermochromic ink powder is contained at approximately 25 wt %.

According to an embodiment of the present invention, based on the total weight of a thermochromic ink composition, it is preferable that the thermochromic ink powder (total sum of first to third thermochromic ink powders) of approximately 10 wt % to approximately 40 wt % and the binder of approximately 60 wt % to approximately 90 wt % are contained.

According to an embodiment of the present invention, the first color exhibited by the first thermochromic ink powder may be yellow, which implements a color at approximately 50° C., the second color exhibited by the second thermochromic ink powder may be cyan, which implements a color at approximately 40° C., and the third color exhibited by the third thermochromic ink powder may be magenta, which implements a color at approximately 35° C.

According to an embodiment of the present invention, the thermochromic Ink powder is prepared by adding a melamine copolymer, styrene maleic anhydride, methyl ester, ethyl ester, and Thermo Dye Ink powders exhibiting the first to third colors.

The thermochromic ink powder has a property of changing colors according to temperature, so that it is possible to change a color to be displayed on the outside of the thermometer by adjusting the mixing ratio of each of the thermal ink powders. It is also possible to adjust a reference temperature at which a color changes by adjusting the mixing ratio of the thermochromic ink powders.

The binder serves to color the thermochromic ink composition and adheres the thermochromic ink composition to the first film layer, and may include a thermosetting binder and a UV curing binder. Examples of a specific binder component may include one or more selected from polyurethane, polyacrylate, polyurethane acrylate, polyurethane acrylic epoxy, polyacrylamide, poly acrylic acid, polyacrylepoxy, polystyrene butadiene, and polysilica, and polyurethane acrylate is most preferred.

According to an embodiment of the present invention, the patch-type thermometer of the present invention may include the second film layer. The second film layer functions to minimize external factors which interfere with temperature measurement when measuring a body temperature. For example, when infants and toddlers play in the water, or in the summer when people sweat a lot, water or sweat components may interfere with accurate body temperature measurement, and the second film layer prevents the above from happening, thereby allowing relatively accurate body temperature measurement. Here, the thickness of the second film layer is preferably in the range of approximately 20 μm to approximately 50 μm.

The second film layer may have a waterproof function for the above purpose. The second film layer may contain one or more components selected from urethane, PE, and PP.

According to an embodiment of the present invention, an adhesive layer is further included between the second film layer and the thermochromic ink composition mixed layer to attach a urethane film layer and a thermochromic ink layer to each other. Examples of the adhesive layer may include one or more selected from polyurethane, polyacrylate, polyurethane acrylate, polyurethane acrylic epoxy, polyacrylamide, poly acrylic acid, polyacrylepoxy, polystyrene butadiene, and polysilica, and polyurethane acrylate is most preferred.

The thickness of the adhesive layer is preferably in the range of approximately 10 μm to approximately 30 μm.

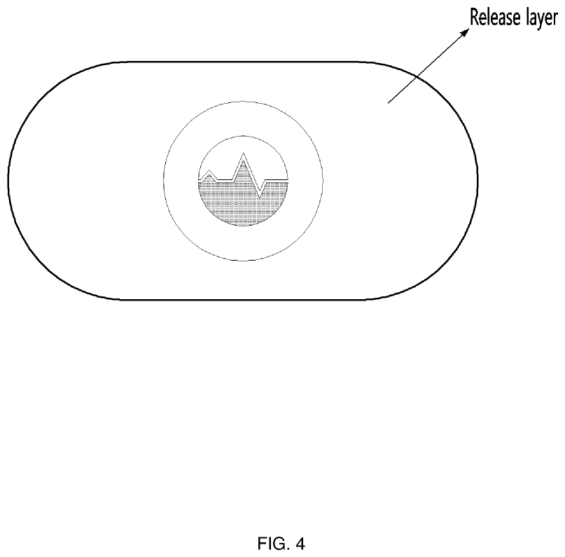

According to an embodiment of the present invention, a removable release layer may be formed on the upper side of the second film layer and on the lower side of the first film layer to protect a final product from external impact. The release layer is to be removed immediately before being attached to a person's body part, and thus, is preferably a film which may be easily removed. The upper and lower release layers may use one or more components selected from PET, PE, PP, and paper. An example in which the release layer is formed is shown in FIG. 4, but is not limited thereto. Here, the thickness of the upper release layer is preferably from approximately 40 μm to approximately 60 μm, and the thickness of the lower release layer is preferably from approximately 50 μm to approximately 80 μm.

According to an embodiment of the present invention, the thickness of a stacked structure including the first film layer, the thermochromic ink composition mixed layer, the adhesive layer, the second film layer, and the upper and lower release layers may preferably be approximately 160 μm to approximately 360 μm, and more preferably approximately 200 μm to approximately 330 μm, in terms of improving the fit to consumers. When the thickness is greater than the above range, a user may feel uncomfortable when the patch-type thermometer is attached to his/her body part, and the adhesion strength of a portion of the patch-type thermometer which has come into contact with the body part is weakened, which is not preferable.

Hereinafter, a principle by which a thermometer according to an embodiment of the present invention changes colors will be described.

The first color, the second color, the third color mean the color of each dye, and a color be exhibited to a user is a mixture of the first color, the second color, and the third color.

When the patch-type thermometer of the present invention including the thermochromic ink composition mixed layer in which the thermochromic ink powders capable of exhibiting the first color, the second color, and the third color are mixed is attached to a human body part such as a wrist, the third color is exhibited in the case of a low body temperature or before the thermometer is attached to a human body (lower than or equal to approximately 35° C.), a mixed color of the first and second colors is exhibited at approximately 35° C. to approximately below 37.5° C., which is a normal body temperature of a person, with the third color gradually disappearing, and only the first color is exhibited with the second color gradually disappearing when the body temperature of a person becomes higher than or equal to approximately 37.5° C. due to high fever. For example, a color to be displayed may be gray at a low body temperature, light green at a normal body temperature, and yellow at a high body temperature.

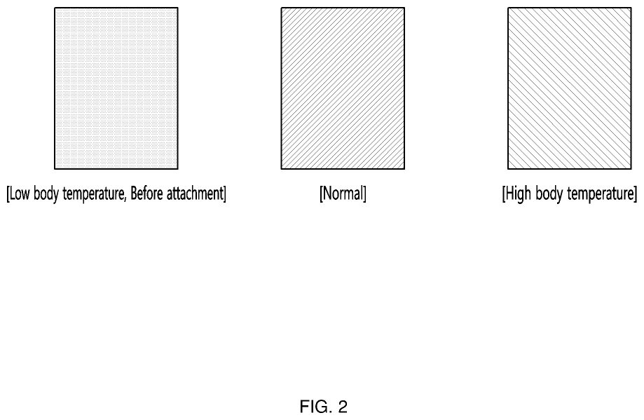

Referring to FIG. 2 of the present disclosure, it is possible to understand how the patch-type thermometer of the present disclosure is implemented before the attachment of the patch-type thermometer to a human body, after the attachment of the same to a human body of a normal body temperature, and when a body temperature is high.

The patch-type thermometer of the present invention may be applied in a variety of forms as long as it may be attached to a human body, in addition to a band-type, and is not limited to the present embodiment of the present disclosure.

A patch-type thermometer of the present invention enables a relatively accurate body temperature measurement without being affected by the external environment even in a variety of environments, and accordingly, even when outside where there is no thermometer, it is possible to be aware of the physical conditions of infants, toddlers, and the elderly through a continuous monitoring by a guardian.

The patch-type thermometer of the present invention is thinner than a typical adhesive-type thermometer, thereby minimizing a person's feeling of rejection to the thermometer being in touch with the skin, and thus, has good adhesion properties and fit to a person's body.

In addition, given the circumstances that it is not always possible to carry a thermometer, the present invention allows a user to easily recognize the change in body temperature only by simple attachment and color checking, so that there is an advantage of not having to carry a thermometer and it is possible to quickly respond to infectious diseases accompanied by a high fever with an accurate temperature check and first aid.

Although the patch type thermometer and application product for same have been described with reference to the specific embodiments, they are not limited thereto. Therefore, it will be readily understood by those skilled in the art that various modifications and changes can be made thereto without departing from the spirit and scope of the present invention defined by the appended claims.

1. A patch-type thermometer comprising:
a first film layer; and
a thermochromic ink composition mixed layer stacked on the first film layer and composed of a thermochromic ink composition including first thermochromic ink powder exhibiting a first color, second thermochromic ink powder exhibiting a second color, third thermochromic ink powder exhibiting a third color, and a binder.

2. The patch-type thermometer of claim 1, further comprising:
an adhesive layer stacked on a lower side of the first film layer; and
a lower release layer stacked on a lower side of the adhesive layer.

3. The patch-type thermometer of claim 1, further comprising:
an adhesive layer stacked on an upper side of the thermochromic ink composition mixed layer; and
a second film layer stacked on an upper side of the adhesive layer.

4. The patch-type thermometer of claim 3, further comprising:
a lower release layer stacked on the lower side of the first film layer; and an upper release layer stacked on an upper side of the second film layer.

5. The patch-type thermometer of claim 4, wherein the upper release layer or the lower release layer contains one or more components selected from PET, PE, PP, and paper.

6. The patch-type thermometer of claim 1, wherein the first film layer contains one or more components selected from an OPP film, PET, PP, and paper.

7. The patch-type thermometer of claim 3, wherein the second film layer is a transparent film.

8. The patch-type thermometer of claim 7, wherein the second film layer contains one or more components selected from urethane, PE, and PP.

9. The patch-type thermometer of claim 1, wherein the first thermochromic ink powder to the third thermochromic ink powder comprise a melamine copolymer, styrene maleic anhydride, methyl ester, ethyl ester, and Thermo Dye Ink powders exhibiting the first to third colors.

10. The patch-type thermometer of claim 1, wherein based on the total weight of the thermochromic ink powders, which is the sum of the weight of each of the first, second, and third thermochromic ink powders, the first thermochromic ink powder exhibiting the first color is contained in a range of approximately 20 wt % to approximately 40 wt %, the second thermochromic ink powder exhibiting the second color is contained in a range of approximately 40 wt % to approximately 60 wt %, the third thermochromic ink powder exhibiting the third color is contained in a range of approximately 20 wt % to approximately 40 wt %, or the first thermochromic ink powder exhibiting the first color is contained in a range of approximately 25 wt % to approximately 35 wt %, the second thermochromic ink powder exhibiting the second color is contained in a range of approximately 45 wt % to approximately 55 wt %, the third thermochromic ink powder exhibiting the third color is contained in a range of approximately 25 wt % to approximately 35 wt %.
